(19) United States
(12) Patent Application Publication
(10) Pub. No.: US 2023/0133135 A1
(43) Pub. Date: May 4, 2023

(54) DISCOVERY OF FIFTEEN NEW ANTI-AGING PLANT EXTRACTS AND IDENTIFICATION OF CELLULAR PROCESSES THEY AFFECT AS NEW CALORIC RESTRICTION MIMETICS

(71) Applicant: IDUNN TECHNOLOGIES, Rosemère (CA)

(72) Inventors: Eric SIMARD, Rosemère (CA); Vladimir TITORENKO, Montréal (CA)

(21) Appl. No.: 17/906,489

(22) PCT Filed: Mar. 19, 2021
(86) PCT No.: PCT/CA2021/050364

§ 371 Date: Sep. 16, 2022

Related U.S. Application Data
(60) Provisional application No. 62/991,717, filed on Mar. 19, 2020.

(51) Int. Cl. A61K 36/889; C12Q 1/02; A61K 36/38; A61K 36/185; A61K 36/53; A61K 36/28; A61K 36/752; A61K 36/87; A61K 36/71; A61K 36/48; A61K 36/29; A61K 36/73; A61K 31/05; A61K 31/155; A61K 31/201; A61K 31/132; A61K 8/9789; A61K 8/9794; A61K 8/34; A61K 8/43; A61K 8/44; A61K 8/41; A61Q 19/08

(52) U.S. Cl. CPC A61K 36/889; C12Q 1/025; A61K 36/38; A61K 36/185; A61K 36/53; A61K 36/28; A61K 36/752; A61K 36/87; A61K 36/71; A61K 36/48; A61K 36/29; A61K 36/73; A61K 31/05; A61K 31/155; A61K 31/201; A61K 31/132; A61K 8/9789; A61K 8/9794; A61K 8/347; A61K 8/43; A61K 8/44; A61K 8/41; A61Q 19/08

(57) ABSTRACT

It is provided an anti-aging composition comprising at least one plant extract and a carrier, the at least one plant extract is at least one of Serenoa repens, Hypericum perforatum, Ilex paraguariensis, Ocimum tenuiflorum, Solidago virgaurea, Citrus sinensis, Humulus lupulus, Vitis vinifera, Andrographis paniculata, Hydrastis canadensis, Trigonella foenum-graecum, Berberis vulgaris, Crataegus monogyna, Taraxacum erythrospermum, Ilex paraguariensis, and a combination thereof.

### CROSS-REFERENCE TO RELATED APPLICATION

The present application is claiming priority from U.S. Provisional Application No. 62/991,717 filed Mar. 19, 2020, the content of which is hereby incorporated by reference in its entirety.

### TECHNICAL FIELD

The present description relates to an anti-aging composition comprising at least one geroprotective plant extract.

### BACKGROUND

It is known that aging drives disease. Nearly every major killer disease in developed countries shares a common feature: the risk of getting the disease increases dramatically as you get older. For example, the likelihood of being diagnosed with Alzheimer's disease doubles every five years after the age of 65. A similar kind of relationship can be seen for most types of cancer, heart disease, diabetes, kidney disease, and many others.

The rate of aging may also be measured, and an accelerated rate of aging may be considered ‘premature aging’, while a slower rate of the aging process may extend health span. It is desirable to maximize the healthy lifespan of cells and organisms and it is also desirable to extend the healthy lifespan by decreasing the rate of aging process and the onset of dysfunctional or disease states. Shortening the lifespan and/or accelerating apoptosis of unhealthy, diseased, damaged, or cancerous cells may also be desirable.

Recent discoveries suggest that aging is neither driven by accumulation of molecular damage of any cause, nor by random damage of any kind. Studies in humans and model organisms aimed at elucidating the molecular mechanisms of aging have demonstrated the existence of broadly conserved longevity pathways, and, for the first time, offer real hope of intervening to enhance healthy aging. The best-characterized intervention for delaying aging is dietary restriction (also referred to as caloric restriction). Many studies have shown that a reduced calorie regimen can increase lifespan and delay the onset of multiple age-related phenotypes in a diverse range of organisms, including the entire major model systems used in biomedical research.

While treatments exist for some symptoms of aging-associated disorders, no treatments are currently known that delay aging of the entire organism by targeting multiple cellular and organismal processes with the help of natural extract(s). In addition, by slowing the rate of aging, it may be possible to delay the onset of various diseases/conditions associated with aging.

Therefore, the implementation of complex mixtures of several (or many) natural products modulating different signaling pathways is desirable to achieve a more important anti-aging effect.

The budding yeast Saccharomyces cerevisiae is a widely used model organism in aging research because it offers three significant advantages in studying mechanisms of aging and longevity. First, S. cerevisiae has relatively short and easily measurable replicative and chronological lifespans. Second, the S. cerevisiae genome has been completely sequenced and many strain collections for yeast genome interrogation are commercially available. Third, S. cerevisiae is amenable to comprehensive molecular analyses that have been used to uncover mechanisms of various cell biological processes. Because of these advantages, studies in S. cerevisiae discovered many genes, signaling pathways and chemical compounds that, following their discovery in budding yeast, were implicated in aging and longevity in organisms across an evolutionary tree. It is therefore commonly believed that the major aspects and underlying mechanisms of aging and aging-associated pathology have been conserved throughout evolution.

It is an aim to understand mechanisms through which certain chemical compounds act as geroprotectors capable of delaying chronological aging and postponing aging-associated pathology in budding yeast. A recent screen of a collection of thirty-five plant extracts (PEs) had identified six PEs that can prolong chronological lifespan (CLS) and delay chronological aging in S. cerevisiae (Lutchman et al., 2016, Oncotarget, 7: 16542-16566). As demonstrated, in S. cerevisiae, the six PEs previously disclosed exhibit different effects on cellular processes known to define longevity in eukaryotes across species. The six PEs extend yeast CLS through different signaling pathways and protein kinases converged into a network; this network is known to define the rate of chronological aging in S. cerevisiae and to regulate longevity in other eukaryotic organisms. The network integrates the pro-aging TORC1 (target of rapamycin complex 1) pathway, the pro-aging PKA (protein kinase A) pathway, the pro-aging PKH1/2 (Pkb-activating kinase homolog) pathway, the anti-aging SNF1 (sucrose non-fermenting) pathway, the anti-aging ATG (autophagy) pathway, the pro-aging serine/threonine-protein kinase Sch9 and the anti-aging serine/threonine-protein kinase Rim15 (regulator of IME2) (Lutchman et al., 2016, Oncotarget, 7: 50845-50863).

It is still desirable to be provided with new aging-delaying (geroprotective) compounds/formulation.

### SUMMARY

It is provided an anti-aging composition comprising at least one geroprotective plant extract and a carrier, the at least one plant extract is at least one of Serenoa repens, Hypericum perforatum, Ilex paraguariensis, Ocimum tenuiflorum, Solidago virgaurea, Citrus sinensis, Humulus lupulus, Vitis vinifera, Andrographis paniculata, Hydrastis canadensis, Trigonella foenum-graecum, Berberis vulgaris, Crataegus monogyna, Taraxacum erythrospermum, Ilex paraguariensis, and a combination thereof.

In an embodiment, composition encompassed herein comprises at least two different anti-aging agents, wherein said at least two anti-aging agents are two different geroprotective plant extracts or one geroprotective plant extract and a second anti-aging agent.

In a further embodiment, the second anti-aging agent is resveratrol, metformin, myriocin, or spermidine.

In another embodiment, the at least one geroprotective plant extract promotes a hormetic stress response.

In an additional embodiment, the at least one geroprotective plant extract lowers the rate of aging.

In an embodiment, the at least one geroprotective plant extract increases rate in mitochondrial respiration, reduces declined in age-related chronology of changes in reactive oxygen species abundance, increases protection of cellular macromolecules from oxidative damage, and/or decreases cell susceptibility to long-term oxidative and thermal stresses.

In a further embodiment, the at least one geroprotective plant extract is from a plant part.

In an embodiment, the plant part is at least one of berry, aerial parts, leaf, herb, fruit, fruit skin, root, seed, root bark, leaf flower, stem, and a combination thereof.

In a further embodiment, the composition encompassed herein comprises between 0.02% to 1.0% (w/v) of the at least one plant extract.

In a further embodiment, the composition encompassed herein is formulated as a cosmetic composition, a dermatological composition, a nutraceutical composition or a pharmaceutical composition.

It is provided the use of the composition described herein, for prolonging longevity of a subject.

In an embodiment, the subject is a human, an animal or a yeast.

It is further provided a method of prolonging longevity of a subject comprising administering to the subject an effective amount of the composition described herein.

### BRIEF DESCRIPTION OF THE DRAWINGS

Reference will now be made to the accompanying drawings.

FIG. 1 illustrates 0.5% (w/v) PE26, 0.5% (w/v) PE39, 0.5% (w/v) PE42, 0.3% (w/v) PE47, 0.3% (w/v) PE59, 0.1% (w/v) PE64, 0.5% (w/v) PE68 and 1.0% (w/v) PE69 exhibit the highest extending effects on the chronological lifespan (CLS) of wild-type (WT) yeast cultured under non-CR conditions on 2% (w/v) glucose. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. Survival curves (the upper panels in A-H) and the mean and maximum lifespans (the lower two panels in A-H) of chronologically aging WT cells cultured without a PE (cells were subjected to ethanol-mock treatment) or with a PE (which was added at the concentration optimal for CLS extension) are shown. Data are presented as means±SEM (n=6). In the upper panels in A-H, CLS extension was significant for each of the PEs tested (p<0.05; the p values for comparing each pair of survival curves were calculated using the logrank test). In the lower two panels in A-H, *p<0.05, **p<0.01, ***p<0.001; the p values for comparing the means of two in groups were calculated using an unpaired two-tailed t test as described in Materials and Methods).

FIG. 2 illustrates that 0.1% (w/v) PE72, 0.3% (w/v) PE75, 0.5% (w/v) PE77, 0.3% (w/v) PE78, 0.5% (w/v) PE79, 0.3% (w/v) PE81 and 0.5% (w/v) PE83 exhibit the highest extending effects on the CLS of WT yeast cultured under non-CR conditions on 2% (w/v) glucose. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. Survival curves (the upper panels in A-G) and the mean and maximum lifespans (the lower two panels in A-G) of chronologically aging WT cells cultured without a PE (cells were subjected to ethanol-mock treatment) or with a PE (which was added at the concentration optimal for CLS extension) are shown. Data are presented as means±SEM (n=6). In the upper panels in A-G, CLS extension was significant for each of the PEs tested (p<0.05; the p values for comparing each pair of survival curves were calculated using the logrank test as described in Materials and Methods). In the lower two panels in A-G, *p<0.05, **p<0.01, ***p<0.001; the p values for comparing the means of two in groups were calculated using an unpaired two-tailed t test).

FIG. 3 illustrates that each of the fifteen PEs extends the longevity of chronologically aging yeast under non-CR conditions on 2% (w/v) glucose significantly more efficiently than it does under CR conditions on 0.5% (w/v) glucose. WT cells were cultured in the synthetic minimal YNB medium initially containing 2% (w/v) or 0.5% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 0.5% (w/v) or 2% (w/v) glucose and 2.5% (v/v) ethanol. The extent to which each of the PE tested increases the mean (A) and maximum (B) CLS under non-CR and CR conditions was calculated based on the data presented in FIGS. 1 and 2, and FIGS. 14 and 15. *p<0.05, **p<0.01, ***p<0.001; the p values for comparing the means of two in groups were calculated using an unpaired two-tailed t test.

FIG. 4 illustrates that each of the fifteen PEs extends the longevity of chronologically aging yeast because it decreases the rate of aging but not because it lowers the baseline mortality rate. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. Survival curves shown in FIGS. 1 and 2 were used to calculate the age-specific mortality rates of chronologically aging WT yeast populations cultured without a PE (cells were subjected to ethanol-mock treatment) or with a PE (which was added at the concentration optimal for CLS extension). The natural logarithms of the mortality rate values for each time point were plotted against days of cell culturing. The values of the age-specific mortality rates, Gompertz slope (also known as the mortality rate coefficient G) and mortality rate doubling time (MRDT) were calculated as described in Materials and Methods. Each of the fifteen longevity-extending PEs caused a substantial decline in the value of G and a considerable rise in the value of MRDT.

FIG. 5 illustrates that each of the fifteen geroprotective PEs stimulates mitochondrial respiration in yeast cultured under non-CR conditions. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. Oxygen uptake by live yeast cells was measured using polarography. Age-related changes in the rate of mitochondrial oxygen consumption are shown. Data are presented as means±SEM (n=3; *p<0.05, **p<0.01, ns, not significant; the p values for comparing the means of two in groups were calculated using an unpaired two-tailed t test).

FIG. 6 illustrates that each of the fifteen geroprotective PEs alters the age-related chronology of changes in intracellular ROS in yeast cultured under non-CR conditions. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. The intracellular concentrations of ROS were measured in live yeast by fluorescence microscopy of dihydrorhodamine 123 staining. Age-related changes in the intracellular concentration of ROS are shown. Data are presented as means±SEM (n=3; *p<0.05, **p<0.01, ns, not significant; the p values for comparing the means of two in groups were calculated using an unpaired two-tailed t test).

FIG. 7 illustrates that each of the fifteen geroprotective PEs decreases the extent of age-related oxidative damage to cellular proteins in yeast cultured under non-CR conditions. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. The concentrations of oxidatively damaged (carbonylated) proteins were measured. Age-related changes in the intracellular concentration (nmoles/mg protein) of carbonylated proteins are shown. Data are presented as means±SEM (n=3; *p<0.05, **p<0.01, ns, not significant; the p values for comparing the means of two in groups were calculated using an unpaired two-tailed t test).

FIG. 8 illustrates that many of the fifteen geroprotective PEs slow the aging-associated buildup of oxidatively impaired membrane lipids in yeast cultured under non-CR conditions. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. The concentrations of oxidatively damaged membrane lipids were measured. Age-related changes in the intracellular concentration (equivalents of nmoles H₂O₂/mg protein) of oxidatively damaged membrane lipids are shown. Data are presented as means±SEM (n=3; *p<0.05, ns, not significant; the p values for comparing the means of two in groups were calculated using an unpaired two-tailed t test).

FIG. 9 illustrates that each of the fifteen geroprotective PEs decreases the frequencies of rib2 and rib3 mutations in mitochondrial DNA (mtDNA) of yeast cultured under non-CR conditions. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. The incidences of spontaneous point mutations in the RIB2 and RIB3 genes of mtDNA were measured. Age-related changes in the frequencies of these mtDNA mutations are shown. Data are presented as means±SEM (n=3; *p<0.05, ns, not significant; the p values for comparing the means of two in groups were calculated using an unpaired two-tailed t test).

FIG. 10 illustrates that PE26, PE39, PE42, PE59, PE64, PE69, PE75, PE78, PE79 and PE81 (but not PE47, PE68, PE72, PE77 or PE83) cause a statistically significant decline in the frequencies of can1 mutations in nuclear DNA (nDNA) of yeast cultured under non-CR conditions. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. The incidences of spontaneous point mutations in the CAN1 gene of nDNA were measured. Age-related changes in the frequencies of these nDNA mutations are shown. Data are presented as means±SEM (n=3; *p<0.05, ns, not significant; the p values for comparing the means of two in groups were calculated using an unpaired two-tailed t test).

FIG. 11 illustrates that each of the fifteen geroprotective PEs makes yeast more resistant to chronic (long-term) oxidative and thermal stresses. WT cells were cultured in the synthetic minimal YNB medium initially containing 2% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 2% (w/v) glucose and 2.5% (v/v) ethanol. Spot assays for examining cell resistance to chronic oxidative (B) and thermal (C) stresses were performed. (A) In control samples, serial 10-fold dilutions of cells recovered on different days of culturing were spotted on plates with solid YEP medium containing 2% (w/v) glucose. All pictures were taken after a 3-d incubation at 30° C. (B) In samples subjected to long-term oxidative stress, serial 10-fold dilutions of cells recovered on different days of culturing were spotted on plates with solid YEP medium containing 2% (w/v) glucose and 5 mM hydrogen peroxide. All pictures were taken after a 3-d incubation at 30° C. (C) In samples subjected to long-term thermal stress, serial 10-fold dilutions of cells recovered on different days of culturing were spotted on plates with solid YEP medium containing 2% (w/v) glucose, incubated at 60° C. for 60 min and then transferred to 30° C. All pictures were taken after a 3-d incubation at 30° C.

FIG. 12 illustrates that none of the fifteen longevity-extending PEs statistically significantly affects glucose consumption by WT yeast cultures under non-CR conditions on 2% (w/v) glucose), wherein WT cells were cultured in the synthetic minimal YNB medium initially containing 2% (w/v) glucose, in the presence of a longevity-extending PE (which was added at an optimal longevity concentration) or its absence.

FIG. 13 illustrates that none of the fifteen longevity-extending PEs statistically significantly alters the growth rate and maximum cell yield of WT yeast cultures under non-CR conditions on 2% (w/v) glucose, wherein WT cells were cultured in the synthetic minimal YNB medium initially containing 2% (w/v) glucose, in the presence of a longevity-extending PE (which was added at an optimal longevity-extending concentration) or its absence.

FIG. 14 illustrates that 0.5% (w/v) PE26, 0.5% (w/v) PE39, 0.5% (w/v) PE42, 0.3% (w/v) PE59 and 0.5% (w/v) PE68 (but not 0.3% (w/v) PE47, 0.1% (w/v) PE64 or 1.0% (w/v) PE69) extend the CLS of WT yeast cultured under CR conditions on 0.5% (w/v) glucose. WT cells were cultured in the synthetic minimal YNB medium initially containing 0.5% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 0.5% (w/v) glucose and 2.5% (v/v) ethanol. Survival curves (the upper panels in A-H) and the mean and maximum lifespans (the lower two panels in A-H) of chronologically aging WT cells cultured without a PE (cells were subjected to ethanol-mock treatment) or with a PE (which was added at the concentration optimal for CLS extension under non-CR conditions) are shown. Data are presented as means±SEM (n=6). In the upper panels in A-C, E and F, CLS extension was significant for each of the PEs tested (p<0.05; the p values for comparing each pair of survival curves were calculated using the logrank test). In the lower two panels in A-C, E and F, **p<0.01, ***p<0.001; the p values for comparing the means of two in groups were calculated using an unpaired two-tailed t test). In the upper panels in D, F and H, CLS extension was statistically not significant for each of the PEs tested (the p values for comparing each pair of survival curves were calculated using the logrank test). In the lower two panels in D, F and H, ns, not significant; the p values for comparing the means of two in groups were calculated using an unpaired two-tailed t test).

FIG. 15 illustrates that 0.3% (w/v) PE78 and 0.5% (w/v) PE83 (but not 0.1% (w/v) PE72, 0.3% (w/v) PE75, 0.5% (w/v) PE77, 0.5% (w/v) PE79 or 0.3% (w/v) PE81) extend the CLS of WT yeast cultured under CR conditions on 0.5% (w/v) glucose. WT cells were cultured in the synthetic minimal YNB medium initially containing 0.5% (w/v) glucose, in the presence of a PE or its absence. In the cultures supplemented with a PE, ethanol was used as a vehicle at a final concentration of 2.5% (v/v). In the same experiment, WT cells were also subjected to ethanol-mock treatment by being cultured in the synthetic minimal YNB medium initially containing 0.5% (w/v) glucose and 2.5% (v/v) ethanol. Survival curves (the upper panels in A-G) and the mean and maximum lifespans (the lower two panels in A-G) of chronologically aging WT cells cultured without a PE (cells were subjected to ethanol-mock treatment) or with a PE (which was added at the concentration optimal for CLS extension under non-CR conditions) are shown. Data are presented as means±SEM (n=6). In the upper panels in D and G, CLS extension was significant for each of the PEs tested (p<0.05; the p values for comparing each pair of survival curves were calculated using the logrank test). In the lower two panels in D and G, **p<0.01; the p values for comparing the means of two in groups were calculated using an unpaired two-tailed t test). In the upper panels in A-C, E and F, CLS extension was statistically not significant for each of the PEs tested (the p values for comparing each pair of survival curves were calculated using the logrank test). In the lower two panels in A-C, E and, ns, not significant; the p values for comparing the means of two in groups were calculated using an unpaired two-tailed t test).

### DETAILED DESCRIPTION

It is provided an anti-aging composition comprising at least one plant extract.

It is also provided an anti-aging composition comprising at least two different anti-aging agents. The anti-aging agents are at least two plant extracts or a combination of one plant extract and a second agent, being for example resveratrol or spermidine. The composition comprising two agents has a superior effect on regulating longevity compared to the use of each individual anti-aging agent.

As described herein, the plant extract is at least one of Serenoa repens, Hypericum perforatum, Ilex paraguariensis, Ocimum tenuiflorum, Solidago virgaurea, Citrus sinensis, Humulus lupulus, Vitis vinifera, Andrographis paniculata, Hydrastis canadensis, Trigonella foenum-graecum, Berberis vulgaris, Crataegus monogyna, Taraxacum erythrospermum, Ilex paraguariensis, and a combination thereof.

In an embodiment, the plant extract is from a part of the plant, such as for example berry, aerial parts, leaf, herb, fruit, fruit skin, root, seed, root bark, leaf flower, stem or a combination thereof.

A robust cell viability assay was used to search for commercially available plant extracts that can substantially prolong the chronological lifespan of budding yeast. Many of these plant extracts have been used in traditional Chinese and other herbal medicines or the Mediterranean and other customary diets. The search led to a discovery of fifteen plant extracts that significantly extend the longevity of chronologically aging yeast not limited in calorie supply. It is provided that each of these longevity-extending plant extracts is a geroprotector that decreases the rate of yeast chronological aging and promotes a hormetic stress response. Each of the fifteen geroprotective plant extracts mimics the longevity-extending, stress-protecting, metabolic and physiological effects of a caloric restriction diet in yeast cells that are not limited in calorie supply. It is further provided that the fifteen geroprotective plant extracts exhibit partially overlapping effects on a distinct set of longevity-defining cellular processes. These effects include a rise in coupled mitochondrial respiration, an altered age-related chronology of changes in reactive oxygen species abundance, protection of cellular macromolecules from oxidative damage, and an age-related increase in the resistance to long-term oxidative and thermal stresses.

Accordingly it is provided fifteen new geroprotective PEs that extend yeast CLS. It is described that each of these new aging-delaying PEs decreases the rate of yeast chronological aging, stimulates a hormetic stress response and regulates a distinct set of longevity-defining cellular processes. As mentioned in Table 1, each of the geroprotective PEs described herein are produced from specific parts of the plant such as leaves, roots, fruit or flowers, and extracted using appropriate extraction solvent ratios (usually a mixed alcohol and water) in order to allow the concentration of the desired active molecules. This extraction step ensures that the active molecules are extracted from the plant and available for consumption in sufficient quantities. The extraction process allows to geroprotective Pes which results in a different composition than the original plant or plant part. Consuming the whole plant could also cause side effects such as intestinal discomfort, headaches or the like, since these are often plants that are not part of the normal human diet. The extraction process allows isolating and concentrating the active molecules making it possible to use it for an oral consumption approach of a finished, commercial product.

As listed in Table 1, fifty-three PEs were screened wherein the origin and properties of these PEs are shown in Table 1. These PEs are believed to have positive effects on human health, and many of them have been used in traditional Chinese and other herbal medicines or the Mediterranean and other long-established diets.

TABLE 1
Properties of plant extracts (Pes) used in screen for Pes that
can prolong longevity of chronologically aging budding yeast

| Abbreviated | The botanical | Plant part used | | A commercial |
|---|---|---|---|---|
| name of a PE | name of a plant | to make a PE | Properties of a PE | source of a PE |
| PE26 | Serenoa repens | Berry | Extraction solvent: carbon | Idunn |
| | | | dioxide. Extract ratio: 15:1. | Technologies |
| | | | Composition: natural extract | |
| | | | (oil) (45-55%). silica (45-55%). | |
| PE38 | Centella | Herb | Extraction solvent: alcohol (50- | Idunn |
| | asiatica | | 70%). water (30-50%). Extract | Technologies |
| | | | ratio: (8-12):1. Composition: | |
| | | | 10% asiaticoside. 30% total | |
| | | | triterpenes. | |
| PE39 | Hypericum | Aerial parts | Extraction solvent: ethanol (60- | Idunn |
| | perforatum | | 80%). water (20-40%). Extract | Technologies |
| | | | ratio: (5-10):1. Composition: | |
| | | | 0.3% hypericin. | |
| PE40 | Boswellia | Resin | Extraction solvent: methanol | Idunn |
| | serrata | | (80%). water (20%). Extract | Technologies |
| | | | ratio: 20:1. Composition: 65% | |
| | | | boswellic acids. | |
| PE41 | Ruscus | Root | Extraction solvent: ethanol (70- | Idunn |
| | aculeatus | | 80%). water (20-30%). Extract | Technologies |
| | | | Ratio: 8:1. Composition: 10% | |
| | | | ruscogenins. | |
| PE42 | Ilex | Leaf | Extraction solvent: water. | Idunn |
| | paraguariensis | | Extract ratio: (3-10):1. | Technologies |
| | | | Composition: 2% caffeine. | |
| PE43 | Schisandra | Berry | Extraction solvent: ethanol | Idunn |
| | chinensis | | (30%). water (70%). Extract | Technologies |
| | | | ratio: 4/1. 1% schizandrins. | |
| PE44 | Cynara | Leaf | Extraction solvent: water. | Idunn |
| | scolynums L. | | Extract ratio: 4:1. | Technologies |
| | | | Composition: >5% cynarin. | |
| PE45 | Allium cepa L. | Bulb skin | Extraction solvent: ethanol | Idunn |
| | | | (70%). water (30%). Extract | Technologies |
| | | | ratio: (20-25):1. | |
| | | | Composition: >5% quercetin | |
| | | | glycoside derivates. | |
| PE46 | Matricaria | Flower | Extraction solvent: ethanol | Idunn |
| | recutita L. | | (80%). water (20%). Extract | Technologies |
| | | | ratio: 5:1. Composition: 3% | |
| | | | apigenins. | |
| PE47 | Ocinnum | Leaf | Extraction solvent: ethanol | Idunn |
| | tenuiflorum | | (90%). water (10%). Extract | Technologies |
| | | | ratio: 10:1. Composition: >5% | |
| | | | ursolic acid. | |
| PE48 | Rhaphanus | Root | Extraction solvent alcohol (60- | Idunn |
| | sativus L. var. | | 80%). water (40-20%). Extract | Technologies |
| | niger | | ratio: 4:1. Composition: | |
| | | | unknown. | |
| PE49 | Rosmarinus | Leaf | Extraction solvent: acetone. | Idunn |
| | officinalis L. | | Extract ratio: (35-50):1. | Technologies |
| | | | Composition: >50% carnosic | |
| | | | acid. | |
| PE50 | Angelica | Root | Extraction solvent: ethanol | Idunn |
| | archangelica L. | | (50%). water (50%). Extract | Technologies |
| | | | ratio: 4:1. Composition: >3% | |
| | | | organic acids. | |
| PE51 | Epimedium | Herb | Extraction solvent: ethanol | Idunn |
| | grandiflorum | | (60%). water (40%). Extract | Technologies |
| | | | ratio: 20:1. Composition: 20% | |
| | | | icariin. | |
| PE52 | Bacopa | Leaf | Extraction solvent: aqueous | Idunn |
| | monnieri | | alcohol. Extract ratio: 10:1. | Technologies |
| | | | Composition: 20% bacosides. | |
| PE53 | Phaseolus | Bean | Extraction solvent: aqueous | Idunn |
| | vulgaris | | alcohol. Extract ratio: 10:1. | Technologies |
| | | | Composition: unknown. | |
| PE54 | Allium sativum | Bulb | Extraction solvent: water. | Idunn |
| | L. | | Extract ratio: 120:1. | Technologies |
| | | | Composition: 4.5% alliin. | |
| PE55 | Morus alba | Leaf | Extraction solvent: ethanol | Idunn |
| | | | (70%). water (30%). Extract | Technologies |
| | | | ratio: 4:1. Composition: 1% 1- | |
| | | | deoxynojirimycin. | |
| PE56 | Saphora | Flower | Extraction solvent: ethanol. | Idunn |
| | Japonica | | water. Extract ratio: unknown. | Technologies |
| | | | Composition: rutin (40%). | |
| | | | quercetin (60%). | |
| PE57 | Morus nigra | Fruit | Extraction solvent: ethanol. | Idunn |
| | | | water. Extract ratio: 4:1. | Technologies |
| | | | Composition: unknown. | |
| PE58 | Magnolia | Bark | Extraction solvent: unknown. | Idunn |
| | officinalis | | Extract ratio: (35-40):1. | Technologies |
| | | | Composition: 40% honokiol. | |
| PE59 | Solidago | Herb | Extraction solvent: ethanol | Idunn |
| | virgaurea | | (30%). water (70%). Extract | Technologies |
| | | | ratio: 4:1. Composition: >2% | |
| | | | flavonoid hyperosides. | |
| PE60 | Astragalus | Root | Extraction solvent: ethanol. | Idunn |
| | membranaceus | | water. Extract ratio: 8:1. | Technologies |
| | | | Composition: 16% | |
| | | | polysaccharides. | |
| PE61 | Lepidium | Root | Extraction solvent: water. then | Idunn |
| | meyenii | | ethanol (96%) and water (4%). | Technologies |
| | | | Extract ratio: (22-27):1. | |
| | | | Composition: 0.6% macamides | |
| | | | and macaenes. | |
| PE62 | Taraxacum | Leaf | Extraction solvent: ethanol (70- | Idunn |
| | officinale | | 80%). water (20-30%). Extract | Technologies |
| | | | ratio: (4-7):1. Composition: 3% | |
| | | | vitexin. | |
| PE63 | Taraxacum | Root | Extraction solvent: ethanol | Idunn |
| | officinale | | (60%). water (40%). Extract | Technologies |
| | | | ratio: 15:1. Composition: 0.3- | |
| | | | 0.4% phenolic acids (chicoric. | |
| | | | chlorogenic and caftaric acids). | |
| PE64 | Citrus sinensis | Fruit | Extraction solvent: unknown. | Idunn |
| | | | Extract ratio: unknown. | Technologies |
| | | | Composition: ≥20% limonene. | |
| PE65 | Piper | Root | Extraction solvent: ethanol | Idunn |
| | methysticum | | (65%). water (35%.). Extract | Technologies |
| | | | ratio: 8:1. Composition: >30% | |
| | | | kavalactones | |
| PE66 | Handroanthus | Bark | Extraction solvent: ethanol | Idunn |
| | chrysotrichus | | (70%). water (30%). Extract | Technologies |
| | | | ratio: (9-15):1. Composition: | |
| | | | unknown. | |
| PE67 | Euterpe | Fruit | Extraction solvent: water. | Idunn |
| | oleracea | | Extract ratio: 20:1. | Technologies |
| | | | Composition: >10% | |
| | | | polyphenols. | |
| PE68 | Humulus | Whole plant | Extraction solvent: unknown. | Idunn |
| | lupulus | | Extract ratio: (5.5-6.5):1. | Technologies |
| | | | Composition: unknown. | |
| PE69 | Vitis vinifera | Grape skin | Extraction solvent: ethanol | Idunn |
| | | | (30%). water (70%). Extract | Technologies |
| | | | ratio: 450:1. | |
| | | | Composition: ≥5% trans- | |
| | | | resveratrol. | |
| PE70 | Vitis vinifera | Grape | Extraction solvent: water (4%). | Idunn |
| | | | ethanol (96%). Extract ratio: | Technologies |
| | | | 200:1. Composition: ≥20% | |
| | | | oligostilbenes. | |
| PE71 | Malus | Grape - Fruit | Extraction solvent: water (5%). | Idunn |
| | domestica - | | ethanol (95%). Extract ratio: | Technologies |
| | Vitis vinifera | | (500-600):1. Composition: ≥95% | |
| | | | polyphenols. | |
| PE72 | Andrographis | Whole plant | Extraction solvent: unknown. | Idunn |
| | paniculata | | Extract ratio: unknown. | Technologies |
| | | | Composition: ≥20% | |
| | | | andrographolides. | |
| PE73 | Oryza sativa | Fermented rice | Extraction solvent: unknown. | Idunn |
| | fermented with | | Extract ratio: unknown. | Technologies |
| | Monascuss | | Composition: ≥20% monacolin | |
| | purpureus yeast | | K. | |
| PE74 | Melissa | Leaf | Extraction solvent: unknown. | Idunn |
| | officinalis | | Extract ratio: 4:1. | Technologies |
| | | | Composition: ≥1% rosmarinic | |
| | | | acid. | |
| PE75 | Hydrastis | Root | Extraction solvent: ethanol | Idunn |
| | canadensis | | (75%). water (25%). Extract | Technologies |
| | | | ratio: (5-7):1. | |
| | | | Composition: ≥5% berberine | |
| | | | and other alkaloids. | |
| PE76 | Polygonum | Root | Extraction solvent: unknown. | Idunn |
| | cuspidatum | | Extract ratio: unknown. | Technologies |
| | | | Composition: ≥20% resveratrol. | |
| PE77 | Trigonella | Seed | Extraction solvent: ethanol | Idunn |
| | foenum- | | (60%). water (40%). Extract | Technologies |
| | graecum | | ratio: (5-8):1. Composition: | |
| | | | 50% saponins. | |
| PE78 | Berberis | Root bark | Extraction solvent: ethanol | Idunn |
| | vulgaris | | (50%). water (50%). Extract | Technologies |
| | | | ratio: (10-12):1. Composition: | |
| | | | 6% berberine. | |
| PE79 | Cratagus | Leaf flower | Extraction solvent: ethanol | Idunn |
| | monogyna | and stem | (80%). water (20%). Extract | Technologies |
| | | | ratio: (3-6):1. Composition: | |
| | | | 1.5% flavonoids. | |
| PE80 | Sophora | Flower bud | Extraction solvent: water. | Idunn |
| | japonica L. | | Extract ratio: (16-20):1. | Technologies |
| | | | Composition: 95% quercetin. | |
| PE81 | Taraxacum | Leaf | Extraction solvent: ethanol (70- | Idunn |
| | erythrospermum | | 80%). water (20-30%). Extract | Technologies |
| | | | ratio: (4-7):1. Composition: 3% | |
| | | | vitexin. | |
| PE82 | NA | NA | Na-RALA Powder. Sodium R- | Idunn |
| | | | lipoate (>80% Total R-lipoic | Technologies |
| | | | Acid) from synthesis. | |
| PE83 | Ilex | Whole plant | Extraction solvent: unknown. | Idunn |
| | paraguariensis | | Extract ratio: unknown. | Technologies |
| | | | Composition: unknown. | |
| PE84 | Vitis vinifera L. | Seed | Extraction solvent: ethanol. | Idunn |
| | | | water. Extract ratio: unknown. | Technologies |
| | | | Composition: 95% polyphenols. | |
| PE85 | Ganoderma | Mushroom | Extraction solvent: unknown. | Idunn |
| | lucidum | body | Extract ratio: unknown. | Technologies |
| | | | Composition: unknown. | |
| PE86 | Panax ginseng | Root | Extraction solvent: unknown. | Idunn |
| | | | Extract ratio: unknown. | Technologies |
| | | | Composition: unknown. | |
| PE87 | Lycium | Whole plant | Extraction solvent: unknown. | Idunn |
| | barbarum | | Extract ratio: unknown. | Technologies |
| | | | Composition: unknown. | |
| PE88 | Hemerocallis | Flower | Extraction solvent: unknown. | Idunn |
| | fulva | | Extract ratio: unknown. | Technologies |
| | | | Composition: unknown. | |
| PE89 | Curcuma L. | Root | Extraction solvent: unknown. | Idunn |
| | | | Extract ratio: unknown. | Technologies |
| | | | Composition: curcumin solid | |
| | | | lipid microparticles to improve | |
| | | | absorption. | |

To conduct the screen, a robust clonogenic cell viability assay for measuring yeast CLS was used (Lutchman et al., 2016, Oncotarget, 7: 16542-16566). In this assay, the wild-type (WT) strain BY4742 was cultured in the synthetic minimal YNB medium initially containing 2% (w/v) glucose. Cells of budding yeast cultured under such non-caloric restriction (non-CR) conditions are known to age chronologically faster than the ones cultured under CR conditions on 0.2% (w/v) or 0.5% (w/v) glucose.

At the time of cell inoculation into the culturing medium, each of the assessed PEs were added at a final concentration ranging from 0.02% (w/v) to 1.0% (w/v). It is described herein that PE40, PE41, PE44, PE50, PE53, PE66, PE73, PE84, PE86 and PE87 do not affect the mean and maximum CLS of WT yeast if exogenously supplemented within this wide range of initial concentrations. In contrast, PE38, PE43, PE45, PE46, PE48, PE49, PE51, PE52, PE54-PE58, PE60-PE63, PE65, PE67, PE70, PE71, PE74, PE76, PE80, PE82, PE85, PE88 and PE89 were cytotoxic at certain concentrations; they decreased the mean and/or maximum CLS of WT yeast if used at the final concentrations in the 0.1 (w/v) to 1.0% (w/v) range.

The screen revealed that fifteen of fifty-three tested PEs statistically significantly increase the mean and maximum CLS of WT yeast cultured under non-CR conditions on 2% (w/v) glucose (FIGS. 1 and 2). Each of these fifteen PEs extended the longevity of chronologically aging WT yeast. The following PEs exhibited the highest longevity-extending effect under non-CR conditions of cell culturing: 0.5% (w/v) PE26 from berries of Serenoa repens (FIG. 1A), 0.5% (w/v) PE39 from aerial parts of Hypericum perforatum (FIG. 1B), 0.5% (w/v) PE42 from leaves of Ilex paraguariensis (FIG. 1C), 0.3% (w/v) PE47 from leaves of Ocimum tenuiflorum (FIG. 1D), 0.3% (w/v) PE59 from the whole plant of Solidago virgaurea (FIG. 1E), 0.1% (w/v) PE64 from fruits of Citrus sinensis (FIG. 1F), 0.5% (w/v) PE68 from the whole plant of Humulus lupulus (FIG. 1G), 1.0% (w/v) PE69 from grape skins of Vitis vinifera (FIG. 1H), 0.1% (w/v) PE72 from the whole plant of Andrographis paniculata (FIG. 2A), 0.3% (w/v) PE75 from roots of Hydrastis canadensis (FIG. 2B), 0.5% (w/v) PE77 from seeds of Trigonella foenum-graecum (FIG. 2C), 0.3% (w/v) PE78 from root barks of Berberis vulgaris (FIG. 2D), 0.5% (w/v) PE79 from leaves, flowers and stems of Crataegus monogyna (FIG. 2E), 0.3% (w/v) PE81 from leaves of Taraxacum erythrospermum (FIG. 2F), and 0.5% (w/v) PE83 from the whole plant of Ilex paraguariensis (FIG. 2G).

None of the fifteen longevity-extending PEs displays a statistically significant effect on glucose consumption during culturing of WT cells under non-CR conditions on 2% (w/v) glucose (FIG. 12). This finding shows that each of these PEs prolongs the longevity of chronologically aging yeast not because it alters the concentration of exogenous glucose and, thus, not because it affects the metabolic rate of this major source of carbon and energy. It was also found that none of the fifteen longevity-extending PEs exhibits a statistically significant effect on the growth rate and maximum cell yield of WT yeast cultures under non-CR conditions (FIG. 13). Based on this observation, it is concluded that each of them extends the longevity of chronologically aging yeast not because it slows cell proliferation and, thus, not because it desensitizes yeast to harmful chemical compounds produced when cells proliferate.

CR without malnutrition is a low-calorie dietary regimen that extends lifespan in many evolutionarily distant organisms and improves healthspan in laboratory rodents and rhesus monkeys. Certain natural chemicals and synthetic drugs have been shown to elicit the CR-like lifespan-extending and healthspan-improving effects even under non-CR conditions (i.e., when calorie supply is not limited). These natural and synthetic chemical compounds are called CR mimetics (CRMs) if they not only extend longevity under non-CR conditions but also if they exhibit three other effects. First, CRMs do not impair food intake. Second, CRMs have CR-like effects on metabolism and physiology. Third, akin to CR, CRMs decrease the susceptibility to diverse stresses. As reported herein, each of the fifteen longevity-extending PEs increases yeast CLS under non-CR conditions on 2% (w/v) glucose (FIGS. 1 and 2) and none of them compromises glucose intake during culturing under these conditions (FIG. 12). Thus, it seems that all these PEs are CRMs. This conclusion is further supported by observations that each of the fifteen longevity-extending PEs exhibits CR-like effects on several aspects of cell metabolism and stress resistance as evidenced herein.

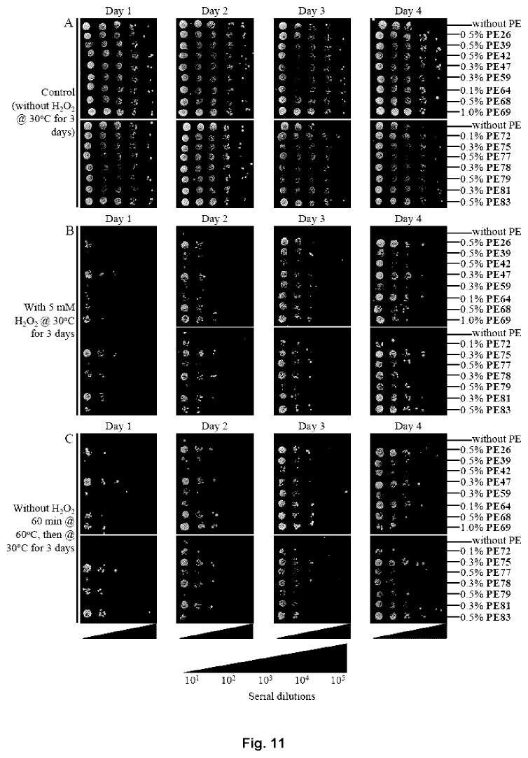

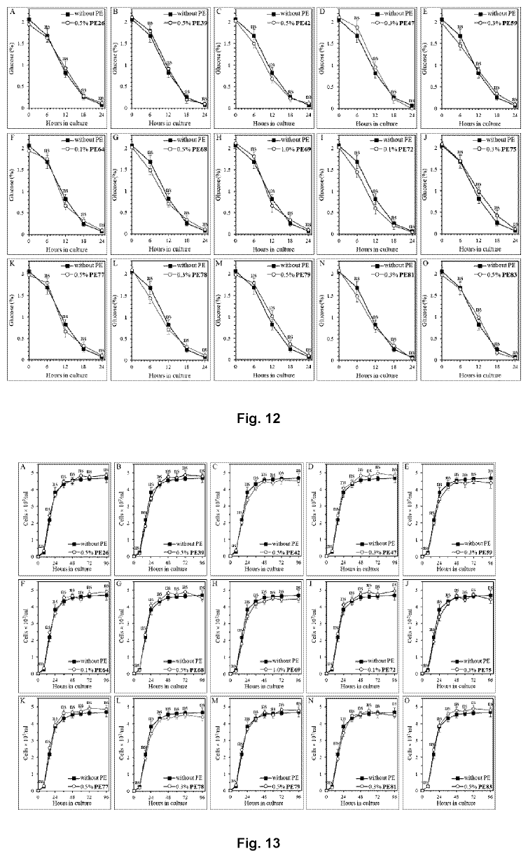

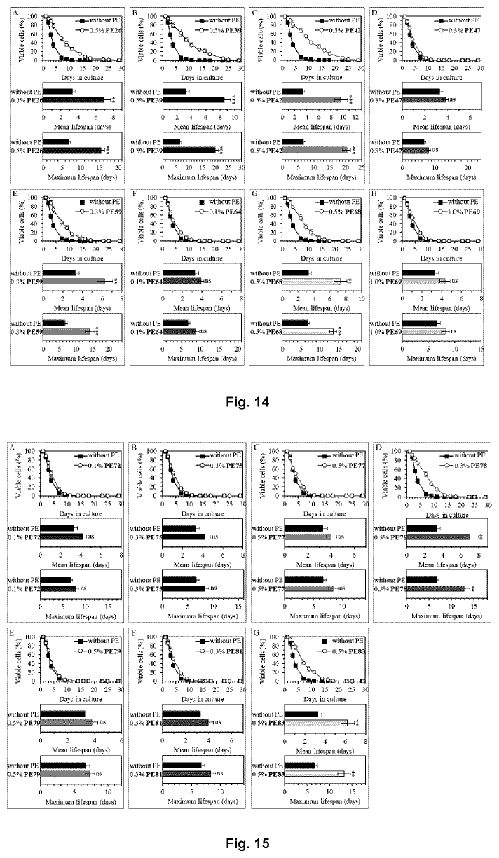

It is known that if the CR diet is administered by culturing yeast in the YNB medium initially containing 0.5% (w/v) glucose, it significantly increases both the mean and maximum CLS of S. cerevisiae. Accordingly, it was investigated how each of the fifteen PEs that extends longevity under non-CR conditions influences the longevity of yeast cultured under CR conditions on 0.5% (w/v) glucose. It was found that eight of the fifteen PEs that prolong the longevity of chronologically aging yeast under non-CR conditions do not increase either the mean or the maximum CLS of S. cerevisiae under CR conditions (FIGS. 14 and 15). These PEs included 0.3% (w/v) PE47 (FIG. 14D), 0.1% (w/v) PE64 (FIG. 14F), 1.0% (w/v) PE69 (FIG. 14H), 0.1% (w/v) PE72 (FIG. 15A), 0.3% (w/v) PE75 (FIG. 15B), 0.5% (w/v) PE77 (FIG. 15C), 0.5% (w/v) PE79 (FIG. 15E) and 0.3% (w/v) PE81 (FIG. 15F). It seems conceivable, therefore, that each of these eight PEs increases yeast CLS because it modulates the same or highly overlapping sets of longevity-defining cellular processes under both CR and non-CR conditions.

Seven of the fifteen PEs that extend yeast longevity under non-CR conditions also increase both the mean and maximum CLS of S. cerevisiae under CR conditions (FIGS. 14 and 15). 0.5% (w/v) PE26 (FIG. 14A), 0.5% (w/v) PE39 (FIG. 14B), 0.5% (w/v) PE42 (FIG. 14C), 0.3% (w/v) PE59 (FIG. 14E), 0.5% (w/v) PE68 (FIG. 14G), 0.3% (w/v) PE78 (FIG. 15D) and 0.5% (w/v) PE83 (FIG. 15G) were among these PEs. Therefore, each of these seven PEs increases yeast CLS under CR conditions because it regulates the sets of longevity-defining cellular processes that differ from (or only partially overlap with) the ones it modulates under non-CR conditions.

The efficiency with which each of the fifteen PEs increases yeast CLS under non-CR conditions was compared to that under CR conditions. The comparison revealed that each of these PEs extends the longevity of chronologically aging yeast under non-CR conditions significantly more efficiently than it does under CR conditions (FIG. 3). This finding shows that each of the fifteen PEs is a more effective longevity-prolonging intervention in chronologically aging yeast not-limited in calorie supply than it is in yeast placed on a CR diet.

Accordingly, each of the fifteen longevity-prolonging PEs is a geroprotector that extends the longevity of chronologically aging yeast because it decreases the rate of aging and stimulates a hormetic stress response. As encompassed herein, the anti-aging composition described herein comprises at least one geroprotector and a carrier, said at least one geroprotector consist of at least one plant extract of Serenoa repens, Hypericum perforatum, Ilex paraguariensis, Ocimum tenuiflorum, Solidago virgaurea, Citrus sinensis, Humulus lupulus, Vitis vinifera, Andrographis paniculata, Hydrastis canadensis, Trigonella foenum-graecum, Berberis vulgaris, Crataegus monogyna, Taraxacum erythrospermum, Ilex paraguariensis, and a combination thereof.

The rate of biological aging at the demographic level depends on the health of a population and can be determined by measuring an age-specific mortality rate of this population. The mortality rates of evolutionarily distant organisms rise with age. The Gompertz mortality function equation can describe this age-related rise in the mortality rate; this equation can be graphically presented as mortality rate data plotted on a semi-log scale against biological age. Geroprotective interventions (also known as geroprotectors) can extend the longevity of organisms across phyla by causing three different effects on the Gompertz mortality function. Some geroprotectors can lower a so-called “baseline” mortality rate by eliciting an equal decline in the mortality rate at any biological age, without affecting a slope of the Gompertz mortality rate. This slope is known as the coefficient G of the age-specific mortality rate; it is inversely proportional to the rate of biological aging. Other geroprotectors can decrease the rate of biological aging because they lower the value of G, thus raising the value of the mortality rate doubling time (MRDT; MRDT=0.693/G). The longevity-extending effects of some other geroprotective interventions can represent a combination of both the drop in the baseline mortality rate and the decline in the value of G (which raises the value of MRDT).

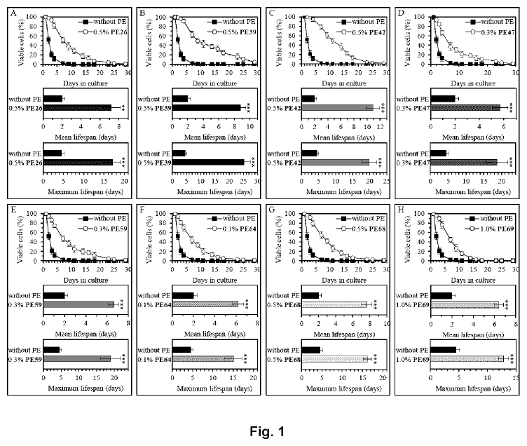

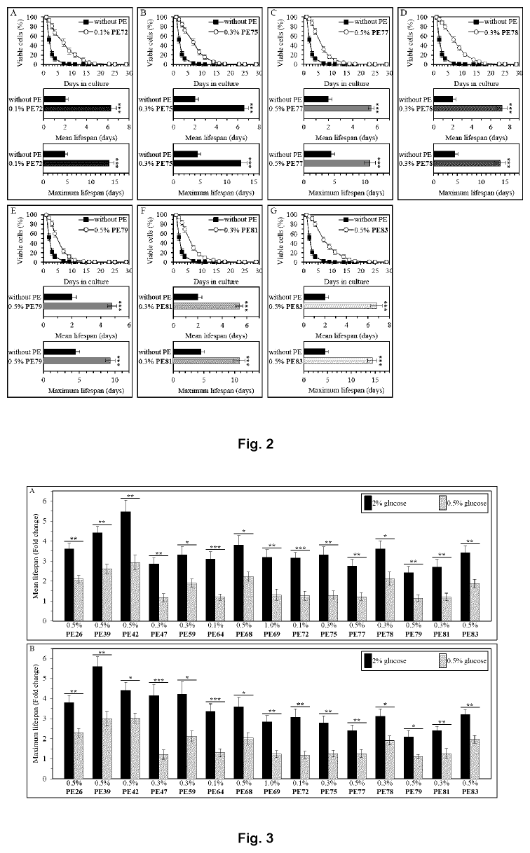

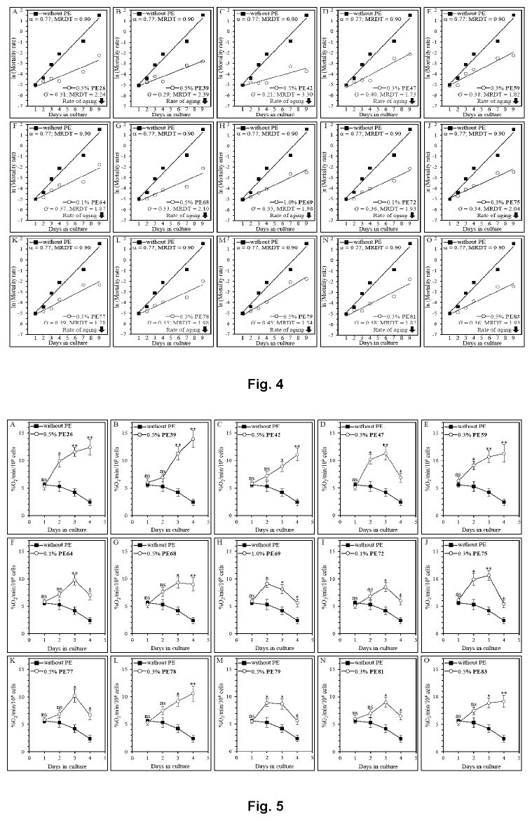

It was further investigated whether each of the fifteen PEs extends yeast longevity by lowering the baseline mortality rate, decreasing the rate of biological aging or by altering both these rates. Therefore, the Gompertz mortality rate analysis of WT cells was conducted under non-CR conditions that were either treated with one of these PEs or subjected to mock treatment. The following was found: 1) none of the fifteen longevity-prolonging PEs affects the baseline mortality rate, and 2) each of them elicits a decline in the coefficient G of the age-specific mortality rate and causes a rise in the value of MRDT (FIG. 4). Based on these observations, each of these PEs is a geroprotector that lengthens the longevity of chronologically aging yeast because it lowers the rate of aging but not because it decreases the baseline mortality rate.

Thus, each of the fifteen longevity-prolonging PEs slows yeast chronological aging because it decreases both the extrinsic and the intrinsic rates of aging. This conclusion is based on findings that each of these PEs extends both the mean and maximum CLS of yeast (FIGS. 1 and 2). The mean lifespans of evolutionarily distant organisms are thought to depend on certain environmental (extrinsic) factors to which cells are exposed before they enter the quiescent or senescent state. In contrast, the maximum lifespans of organisms across species are considered to rely on certain cellular and organismal longevity modifiers that operate after cells enter the quiescent or senescent state.

The data also show that the ability of each of the fifteen longevity-prolonging PEs to decelerate yeast chronological aging correlates with (and is possibly caused by) its ability to elicit a “hormetic” stress response. A characteristic feature of such a response is a nonlinear and biphasic (i.e., inverted U-shaped or J-shaped) dose-response curve. As found, the curves that reflect relationships between PE concentrations and mean or maximum yeast CLS are inverted U-shaped or J-shaped for all these PEs.

It is further demonstrated that each of the fifteen geroprotective PEs intensifies mitochondrial respiration and alters the pattern of age-related changes in intracellular ROS. A distinct set of cellular processes is known to define the rate of yeast chronological aging. These processes include coupled mitochondrial respiration. It was investigated how each of the fifteen geroprotective PEs influences an age-related chronology of changes in coupled mitochondrial respiration, which we measured as the rate of oxygen consumption by yeast cells. It was found that each of these PEs causes a statistically significant increase in the rate of mitochondrial respiration on days 3 and 4 of culturing in the YNB medium initially containing 2% (w/v) glucose (FIG. 5). On these days of culturing in the YNB medium with 2% (w/v) glucose, yeast cells are known to enter and proceed through a stationary (ST) phase of culturing.

The fifteen geroprotective PEs belong to two different groups regarding their effects on the age-related dynamics of changes in coupled mitochondrial respiration under non-CR conditions. The first group of these PEs includes PE47, PE64, PE69, PE72, PE75, PE77, PE79 and PE81. Although all these geroprotective PEs allowed the yeast to maintain the rates of mitochondrial respiration significantly exceeding those in yeast subjected to ethanol-mock treatment, none of them prevented an age-related decline in mitochondrial respiration during the ST phase of culturing (FIGS. 5D, 5F, 5H, 5I, 5J, 5K, 5M and 5N). Of note, all geroprotective PEs from the first group were able to extend yeast CLS only under non-CR conditions on 2% (w/v) glucose (FIGS. 1D, 1F, 1H, 2A, 2B, 2C, 2E and 2F) but not under CR conditions on 0.5% (w/v) glucose (FIGS. 14D, 14F, 14H, 15A, 15B, 15C, 15E and 15F). The second group of geroprotective PEs includes PE26, PE39, PE42, PE59, PE68, PE78 and PE83. These PEs increased the rate of mitochondrial respiration and sustained it high in ST-phase cultures that were recovered on day 4 (FIGS. 5A, 5B, 5C, 5E, 5G, 5L and 5O). Noteworthy, all geroprotective PEs from the second group were able to extend yeast CLS under both non-CR conditions on 2% (w/v) glucose (FIGS. 1A, 1B, 1C, 1E, 1G, 2D and 2G) and CR conditions on 0.5% (w/v) glucose (FIGS. 14A, 14B, 14C, 14E, 14G, 15D and 15G).

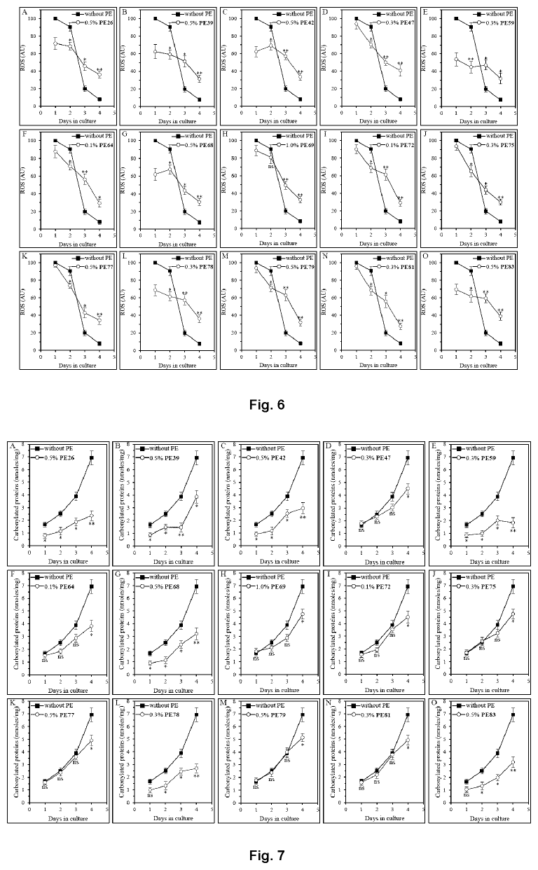

The primary by-products of coupled mitochondrial respiration are several ROS. These ROS of mitochondrial origin are known for their essential roles in defining the rate of aging in organisms across species, including S. cerevisiae. It was found that all fifteen geroprotective PEs alter the age-related dynamics of changes in intracellular ROS (FIG. 6). Each of these PEs slowed an age-related decline in intracellular ROS on days 3 and 4 of culturing, thus enabling a moderate but statistically significant rise in intracellular ROS during the ST phase (FIG. 6). During the post-diauxic (PD) phase on day 2 of culturing, most of the fifteen geroprotective PEs (other than PE69; FIG. 6H) elicited a modest but statistically decline in intracellular ROS (FIG. 6).

Noteworthy, as described herein, there are two different groups of geroprotective PEs with respect to their effects on intracellular ROS during the logarithmic (L) phase on day 1.

PE47, PE64, PE69, PE72, PE75, PE77, PE79 and PE81 did not elicit a substantial change in intracellular ROS during the L phase of culturing on day 1 (FIGS. 6D, 6F, 6H, 6I, 6J, 6K, 6M and 6N). All of them extended yeast CLS only under non-CR conditions on 2% (w/v) glucose (FIGS. 1D, 1F, 1H, 2A, 2B, 2C, 2E and 2F) but not under CR conditions on 0.5% (w/v) glucose (FIGS. 14D, 14F, 14H, 15A, 15B, 15C, 15E and 15F).

In contrast, PE26, PE39, PE42, PE59, PE68, PE78 and PE83 caused a substantial decline in intracellular ROS during the L phase of culturing on day 1 (FIGS. 6A, 6B, 6C, 6E, 6G, 6L and 6O). All these geroprotective PEs stimulated mitochondrial respiration and sustained it high in ST-phase cultures (FIGS. 5A, 5B, 5C, 5E, 5G, 5L and 5O). All of them were also capable of prolonging yeast CLS under both non-CR conditions on 2% (w/v) glucose (FIGS. 1A, 1B, 1C, 1E, 1G, 2D and 2G) and CR conditions on 0.5% (w/v) glucose (FIGS. 14A, 14B, 14C, 14E, 14G, 15D and 15G).

Each of the fifteen geroprotective PEs decreases the extent of age-related oxidative damage to cellular proteins, and many of them slow the aging-associated buildup of oxidatively impaired membrane lipids as well as mitochondrial and nuclear DNA. An age-related rise in the intracellular ROS above a toxic threshold has been shown to cause oxidative damage to cellular proteins, lipids and nucleic acids. The aging-associated accumulation of these oxidized macromolecules is one of the essential contributors to the aging process in yeast and other organisms.

Each of the fifteen geroprotective PEs perturbed the age-related chronology of changes in intracellular ROS (see above). Therefore, it was investigated whether each of them also influences the aging-associated accumulation of oxidatively impaired proteins, lipids and DNA in yeast cells cultured under non-CR conditions on 2% (w/v) glucose.

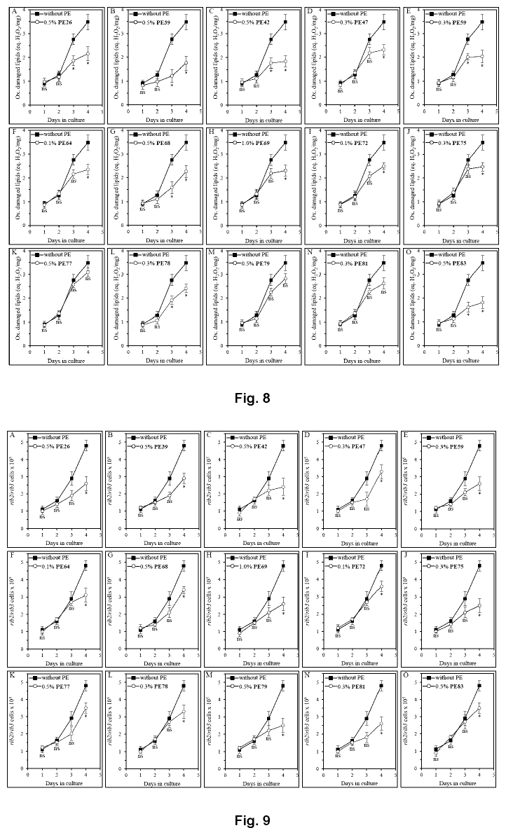

It was found that all fifteen geroprotective PEs elicit a statistically significant decline in the abundance of oxidatively damaged (carbonylated) cellular proteins in ST-phase cultures recovered on day 4 (FIG. 7).

It was noticed that these geroprotective PEs belong to two different groups regarding their effects on the extent of protein carbonylation in yeast cells taken on day 1, 2 or 3 of culturing.

The first group of these PEs includes PE47, PE64, PE69, PE72, PE75, PE77, PE79 and PE81, all of which did not cause a statistically significant decline in the abundance of oxidatively damaged proteins within yeast cells recovered on day 1, 2 or 3 of culturing (FIGS. 7D, 7F, 7H, 7I, 7J, 7K, 7M and 7N). All geroprotective PEs from the first group extended yeast CLS only under non-CR conditions on 2% (w/v) glucose (FIGS. 1D, 1F, 1H, 2A, 2B, 2C, 2E and 2F) but not under CR conditions on 0.5% (w/v) glucose (FIGS. 14D, 14F, 14H, 15A, 15B, 15C, 15E and 15F).

The second group of geroprotective PEs includes PE26, PE39, PE42, PE59, PE68, PE78 and PE83, all of which substantially lowered the abundance of oxidatively damaged proteins in yeast recovered on day 1, 2 or 3 of culturing (FIGS. 7A, 7B, 7C, 7E, 7G, 7L and 7O). Only for PE78 and PE83 such effects on protein carbonylation were not statistically significant in yeast taken on day 1 of culturing (FIGS. 7L and 7O). All geroprotective PEs from the second group increased yeast CLS under both non-CR conditions on 2% (w/v) glucose (FIGS. 1A, 1B, 1C, 1E, 1G, 2D and 2G) and CR conditions on 0.5% (w/v) glucose (FIGS. 14A, 14B, 14C, 14E, 14G, 15D and 15G).

The analysis of how each of the fifteen geroprotective PEs influences the extent of oxidative damage to membrane lipids revealed that PE26, PE39, PE42, PE47, PE59, PE64, PE68, PE69, PE72, PE75, PE78 and PE83 statistically significantly decrease it in ST-phase cultures recovered on day 4 (FIGS. 8A-8J, 8L and 8O). For PE77, PE79 and PE81, a decline in the abundance of oxidatively impaired membrane lipids in yeast cells taken on day 4 of culturing was noticeable but not statistically significant (FIGS. 8K, 8M and 8N). It was found that only those of the fifteen geroprotective PEs that extend yeast CLS under both non-CR and CR conditions significantly lower the abundance of oxidized membrane lipids even in yeast recovered on day 3 of culturing on 2% (w/v) glucose (FIGS. 8A, 8B, 8C, 8E, 8G, 8L and 8O for PE26, PE39, PE42, PE59, PE68, PE78 and PE83). In contrast, a decline in the abundance of oxidatively damaged membrane lipids on day 3 of culturing on 2% (w/v) glucose was noticeable but not statistically significant for any of the geroprotective PEs that increased yeast CLS only under non-CR conditions (FIGS. 8D, 8F, 8H, 8I, 8J, 8K, 8M and 8N for PE47, PE64, PE69, PE72, PE75, PE77, PE79 and PE81).

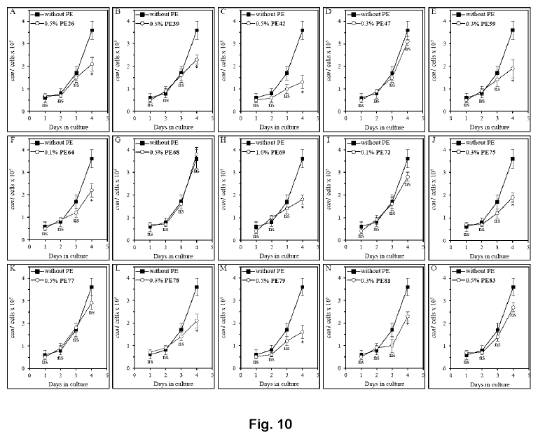

It was then examined how each of the fifteen geroprotective PEs influences the extent of oxidative damage to mitochondrial DNA (mtDNA) and nuclear DNA (nDNA). The oxidative damage to each of these two types of DNA molecules is known to cause an aging-associated buildup of mutations in mtDNA and nDNA. Therefore, the effect of each of the fifteen geroprotective PEs on the frequencies of spontaneous point mutations were investigated in the RIB2 and RIB3 genes of mtDNA as well as the frequencies of spontaneous point mutations in the CAN1 gene of nDNA. All fifteen geroprotective PEs statistically significantly decrease the incidences of rib2 and rib3 mutations in mtDNA of yeast recovered from the ST phase on day 4 but not on any other day of culturing (FIG. 9). Furthermore, PE26, PE39, PE42, PE59, PE64, PE69, PE75, PE78, PE79 and PE81 caused a statistically significant decline in the frequencies of cant mutations in nDNA of yeast cells that were taken from the ST phase on day 4 of culturing only (FIGS. 10A-10C, 10E, 10F, 10H, 10J and 10L-10N). In contrast, neither PE47, PE68, PE72, PE77 nor PE83 elicited a significant change in the incidences of these mutations in nDNA of yeast recovered on any day of culturing (FIGS. 10D, 10G, 10I, 10K and 10O, respectively).

It is further demonstrated that each of the fifteen geroprotective PEs increases cell resistance to long-term oxidative and thermal stresses. Genetic, dietary and chemical interventions that decrease cell susceptibility to chronic (long-term) oxidative and/or thermal stresses have been shown to decelerate the aging process and extend longevity in yeast and other organisms across species. Therefore, the effect of each of the fifteen geroprotective PEs on the susceptibility of chronologically aging yeast cells to these two types of chronic stresses were investigated.

To examine aging-associated changes in cell susceptibility to these long-term stresses, aliquots of yeast cells were recovered on days 1, 2, 3 and 4 of culturing under non-CR conditions in liquid YNB medium with 2% (w/v) glucose. To assess cell susceptibility to chronic oxidative stress, serial dilutions of these cell aliquots were spotted on solid YEP medium with 2% (w/v) glucose and 5 mM hydrogen peroxide and incubated them for 3 days. To assess cell susceptibility to chronic thermal stress, serial dilutions of these cell aliquots were spotted on solid YEP medium with 2% (w/v) glucose, incubated at 60° C. for 60 min, transferred the plates to 30° C. and incubated at this temperature for 3 days.

It was found that each of the fifteen geroprotective PEs makes yeast cells more resistant to chronic oxidative and thermal stresses, especially cells in ST-phase cultures recovered on days 3 and 4 (FIGS. 11B and 11C, respectively).

Accordingly, it is provided fifteen PEs that extend the longevity of chronologically aging budding yeast. All these PEs originate from plants used in traditional Chinese and other herbal medicines or the Mediterranean and other long-established diets. However, none of these PEs had been previously known for their ability to prolong lifespan in yeast or other organisms.

Each of the fifteen longevity-extending PEs encompassed herein prolongs yeast CLS not because it slows the metabolism of glucose, the only source of carbon and energy added to the growth medium. It is revealed that the longevity-extending ability of each of the fifteen PEs is not caused by its negative effect on the proliferation of yeast cells. Thus, it seems likely that none of these PEs can prolong yeast CLS because it slows the formation and release of harmful products of cell proliferation.

The present disclosure provides evidence that each of the fifteen longevity-extending PEs satisfies all the criteria previously proposed for a CRM. CRMs are chemical interventions that can mimic the CR-like lifespan-increasing and healthspan-improving effects even if calorie supply is not limited. First, each of the fifteen PEs prolongs yeast CLS under non-CR conditions. Second, none of these PEs impairs glucose uptake and metabolism. Third, each of them exhibits CR-like effects on specific aspects of metabolism and physiology; these effects include an increased rate of coupled mitochondrial respiration, an altered chronology of changes in intracellular ROS, and a decline in the oxidative damage to cellular proteins, membrane lipids and mtDNA. Fourth, each of them makes cells more resistant to long-term oxidative and thermal stresses. Of note, PE26, PE39, PE42, PE59, PE68, PE78 and PE83 can prolong yeast CLS even under CR conditions, when all cellular processes that limit longevity under non-CR conditions are likely to be suppressed. Therefore, it seems conceivable that each of these seven PEs may stimulate the longevity-extending cellular processes and/or may suppress the longevity-shortening cellular processes that operate only under CR conditions.

The analyses of the Gompertz mortality rates and dose-response curves demonstrated that first, each of the fifteen PEs prolongs yeast CLS because it is a geroprotective agent that decreases the rate of chronological aging but has no effect on the baseline mortality rate. Second, each of these PEs promotes a hormetic stress response in chronologically aging yeast.

Accordingly, it is provided that the fifteen geroprotective PEs described herein differently affect three groups of cellular processes in chronologically aging yeast, as summarized below.

First, each of the fifteen geroprotective PEs significantly increases the rate of coupled mitochondrial respiration and slows a decline in intracellular ROS (known to be the primary products of mitochondrial respiration) within yeast cells that enter and proceed through the ST phase of culturing.

Second, each of them substantially suppresses oxidative damage to cellular proteins and mtDNA in ST-phase yeast cells that enter day 4 of culturing. It was noticed that twelve of these geroprotective PEs also significantly decrease oxidative damage to membrane lipids in ST-phase yeast cells on day 4, whereas PE77, PE79 and PE81 cause a statistically insignificant decline in oxidized membrane lipids within these cells. IT was also found that ten of these geroprotective PEs significantly reduce oxidative damage to nDNA, while neither PE47, PE68, PE72, PE77 nor PE83 exhibits such effect on nDNA.

Third, each of them significantly decreases cell susceptibility to long-term oxidative and thermal stresses, especially the susceptibility of yeast cells that enter and proceed through the ST phase of culturing.

Health Canada government agency defines thirteen of the fifteen geroprotective PEs described here as ones that are safe for human consumption. The agency recommends using eight of them as health-improving supplements with clinically proven benefits to human health. Among these health-improving PEs are PE26, PE47, PE59, PE64, PE69, PE75, PE77 and PE83. For each of them, Health Canada provides a detailed description of source material, routes of administration, doses and dosage forms, uses or purposes, durations of use, risk information, cautions and warnings, contraindications, known adverse reactions, non-medicinal ingredients, specifications, references cited and reviewed, examples of appropriate dosage preparations, and frequencies of use.

### Example I

Yeast Strains Growth and Testing

The wild-type (WT) strain Saccharomyces cerevisiae BY4742 (MATα his3Δ1 1eu2Δ0 lys2Δ0 ura3Δ0) and single-gene-deletion mutant strains in the BY4742 genetic background (all from Thermo Scientific/Open Biosystems) were grown in a synthetic minimal YNB medium (0.67% (w/v) Yeast Nitrogen Base without amino acids from Fisher Scientific; #DF0919-15-3) initially containing 2% (w/v) or 0.5% (w/v) glucose (#D16-10; Fisher Scientific), 20 mg/I L-histidine (#H8125; Sigma), 30 mg/I L-leucine (#L8912; Sigma), 30 mg/I L-lysine (#L5501; Sigma) and 20 mg/l uracil (#U0750; Sigma), with a PE or without it. A stock solution of each PE in ethanol was made on the day of adding this PE to cell cultures. For each PE, the stock solution was added to growth medium with 2% (w/v) or 0.5% (w/v) glucose immediately following cell inoculation into the medium. In a culture supplemented with a PE, ethanol was used as a vehicle at the final concentration of 2.5% (v/v). In the same experiment, yeast cells were also subjected to ethanol-mock treatment by being cultured in growth medium initially containing 2% (w/v) or 0.5% (w/v) glucose and 2.5% (v/v) ethanol. Cells were cultured at 30° C. with rotational shaking at 200 rpm in Erlenmeyer flasks at a “flask volume/medium volume” ratio of 5:1.

A sample of cells was taken from a culture at a certain day following cell inoculation and PE addition into the medium. A fraction of the sample was diluted to determine the total number of cells using a hemacytometer. Another fraction of the cell sample was diluted, and serial dilutions of cells were plated in duplicate onto YEP medium (1% (w/v) yeast extract, 2% (w/v) peptone; both from Fisher Scientific; #BP1422-2 and #BP1420-2, respectively) containing 2% (w/v) glucose (#D16-10; Fisher Scientific) as carbon source. After 2 d of incubation at 30° C., the number of colony-forming units (CFU) per plate was counted. The number of CFU was defined as the number of viable cells in a sample. For each culture, the percentage of viable cells was calculated as follows: (number of viable cells per ml/total number of cells per ml)×100. The percentage of viable cells in the mid-logarithmic growth phase was set at 100%.

The age-specific mortality rate, Gompertz slope or mortality rate coefficient (G) and mortality rate doubling time (MRDT) were calculated as known. The value of the mortality rate was calculated as the number of cells that lost viability (i.e. are unable to form a colony on the surface of a solid nutrient-rich medium) during each time interval divided by the number of viable (i.e. clonogenic) cells at the end of the interval. The natural logarithms of the mortality rate values for each time point were plotted against days of cell culturing. The coefficient G of the age-specific mortality rate was calculated as the slope of the Gompertz mortality line, whereas the value of MRDT was calculated as 0.693/G.

Statistical analysis was performed using Microsoft Excel's Analysis ToolPack-VBA. All data on cell survival are presented as mean±SEM. The p values for comparing the means of two groups using an unpaired two-tailed t-test were calculated with the help of the GraphPad Prism 7 statistics software. The logrank test for comparing each pair of survival curves was performed with GraphPad Prism 7. Two survival curves were considered statistically different if the p value was less than 0.05.

While the description has been described in connection with specific embodiments thereof, it will be understood that it is capable of further modifications and this application is intended to cover any variations, uses, or adaptations and including such departures from the present disclosure as come within known or customary practice within the art and as may be applied to the essential features hereinbefore set forth, and as follows in the scope of the appended claims.

What is claimed is:

1. An anti-aging composition comprising at least one geroprotective plant extract and a carrier, said at least one geroprotective plant extract is at least one of Serenoa repens, Hypericum perforatum, Ilex paraguariensis, Ocimum tenuiflorum, Solidago virgaurea, Citrus sinensis, Humulus lupulus, Vitis vinifera, Andrographis paniculata, Hydrastis canadensis, Trigonella foenum-graecum, Berberis vulgaris, Crataegus monogyna, Taraxacum erythrospermum, Ilex paraguariensis, and a combination thereof.

2. The anti-aging composition of claim 1, comprising at least two different anti-aging agents, wherein said at least two anti-aging agents are two different geroprotective plant extracts or one geroprotective plant extract and a second anti-aging agent.

3. The anti-aging composition of claim 2, wherein the second anti-aging agent is resveratrol, metformin, myriocin, or spermidine.

4. The anti-aging composition of claim 1, wherein the at least one geroprotective plant extract promotes a hormetic stress response.

5. The anti-aging composition of claim 1, wherein the at least one geroprotective plant extract lowers the rate of aging.

6. The anti-aging composition of claim 1, wherein the at least one geroprotective plant extract increases rate in mitochondrial respiration, reduces declined in age-related chronology of changes in reactive oxygen species abundance, increases protection of cellular macromolecules from oxidative damage, and/or decreases cell susceptibility to long-term oxidative and thermal stresses.

7. The anti-aging composition of claim 1, wherein the at least one geroprotective plant extract is from a plant part.

8. The anti-aging composition of claim 7, wherein the plant part is at least one of berry, aerial parts, leaf, herb, fruit, fruit skin, root, seed, root bark, leaf flower, stem, and a combination thereof.

9. The anti-aging composition of claim 1, comprising between 0.02% to 1.0% (w/v) of the at least one geroprotective plant extract.

10. The anti-aging composition of claim 1, formulated as a cosmetic composition, a dermatological composition, a nutraceutical composition or a pharmaceutical composition.

11-12. (canceled)

13. A method of prolonging longevity of a subject comprising administering to said subject an effective amount of the composition of claim 1.

14. The method of claim 13, wherein the subject is a human, an animal or a yeast.
